(19) United States
(12) Patent Application Publication
(10) Pub. No.: US 2024/0139171 A1
(43) Pub. Date: May 2, 2024

(54) TREATMENT AND/OR REDUCTION OF OCCURRENCE OF MIGRAINE

(71) Applicant: CGRP Diagnostics GmbH, Basel (CH)

(72) Inventor: Mark HASLETON, Raanana (IL)

(21) Appl. No.: 18/279,465

(22) PCT Filed: Mar. 2, 2022
(86) PCT No.: PCT/IB2022/051820

§ 371 Date: Aug. 30, 2023

Related U.S. Application Data
(60) Provisional application No. 63/238,448, filed on Aug. 30, 2021.
(60) Provisional application No. 63/155,310, filed on Mar. 2, 2021.

(51) Int. Cl. A61K 31/4545; A61K 31/496; A61K 31/498; A61K 31/517; A61K 31/55; A61K 31/5513; A61P 25/06

(52) U.S. Cl. CPC A61K 31/4545; A61K 31/496; A61K 31/498; A61K 31/517; A61K 31/55; A61K 31/5513; A61P 25/06

(57) ABSTRACT

There is described a method of treating migraine in certain subjects suffering from migraine, by administration of a gepant to the subject. Also described is a method of reducing the incidence of migraine in certain subjects suffering from migraine, by administration of a gepant to the subject. Also described are methods for identifying the certain subjects. Other embodiments are also disclosed.

### RELATED APPLICATIONS

This application claims Paris Convention priority from, and the US benefit of, US provisional patent applications No. 63/238,448, filed 30 Aug. 2021 and titled “Treatment of Migraine”, and 63/155,310, filed 2 Mar. 2021, and titled, “Method of Treating Migraine”. The contents of these two provisional applications are incorporated herein by reference.

### BACKGROUND

Small molecules belonging to the class of gepants have been found to be effective in reducing the frequency of chronic migraine (Lipton R B et al Cephalgia 38:2S 18-9; Dodick D W et al N Engl J Med, 381 (23) (2019), pp. 2230-2241; Goadsby P J Neurology 92 (15 Supplement) (2019), Article S17.001). However, while gepants have been found effective in treating certain headaches, patients can respond in varying ways. For example, a gepant can be totally effective, partially effective, or not effective at all in treating the headache or preventing the occurrence of a headache. It could benefit patient care, conserve physician time, and prevent unnecessary use of a particular course of treatment if it could be determined prior to treatment with a gepant whether use of that antibody will be effective to treat a headache and/or to prevent development of a headache.

Therefore, methods for determining whether treatment comprising a gepant will be effective in the treatment of a patient who has headache or who is susceptible to headache are needed.

### SUMMARY

The present invention relates to methods of treating migraine in a subject comprising determining or having determined whether the subject exhibits allodynia and/or hyperalgesia during the interictal phase of a migraine, and administering a gepant to the subject that does not exhibit signs of allodynia and/or hyperalgesia during the interictal phase of the migraine.

The present invention also relates to methods of treating migraine in a subject comprising determining or having determined whether the subject exhibits allodynia and/or hyperalgesia during the interictal phase of a migraine, and administering a gepant to the subject that does not exhibit allodynia and/or hyperalgesia during the ictal phase of the migraine.

The present invention also relates to methods of treating migraine in a subject comprising determining or having determined whether the subject exhibits allodynia and/or hyperalgesia during the ictal phase of a migraine, and administering a gepant to the subject that does not exhibit allodynia and/or hyperalgesia during the ictal phase of the migraine.

### DETAILED DESCRIPTION

Provided herein is a method of treating migraine in a subject comprising determining or having determined whether the subject exhibits allodynia and/or hyperalgesia during the interictal phase of a migraine, and administering a gepant to the subject that does not exhibit signs of allodynia and/or hyperalgesia during the interictal phase of the migraine.

A large body of evidence supports an important role for CGRP in the pathophysiology of migraine. This evidence gave rise to a global effort to develop a new generation of therapeutics that reduces the availability of CGRP in migraineurs. Recently, humanized monoclonal anti-CGRP antibodies and gepants have been found to be effective in reducing the frequency of chronic or episodic migraine.

Single-unit extracellular recording techniques were used to determine the effects of the monoclonal anti-CGRP antibody fremenezumab (30 mg/kg IV) and its isotype (control) on spontaneous and evoked activity in naive and CSD-sensitized central trigeminovascular neurons in the medullary and upper cervical dorsal horn in anesthetized male and female rats (see, e.g., Example 1).

The study described herein demonstrates that the fremanezumab inhibits naive high-threshold (HT) but not wide dynamic range (WDR) trigeminovascular neurons, that the inhibitory effects are limited to their activation from the intracranial dura but not facial skin or cornea, and that when given sufficient time, this drug prevents activation and sensitization of HT but not WDR neurons by cortical spreading depression. This inhibition was similar in male and female rats. For patients whose chronic and episodic migraines are relieved by anti-CGRP active agents, the findings raise the possibility that HT neurons play a critical previously-unrecognized role in the initiation and chronification of the perception of headache, whereas WDR neurons contribute to the associated allodynia and central sensitization (see Example 1). Clinically, the findings may help explain the therapeutic effects of such agents in reducing headaches of intracranial origin such as migraine, and headaches attributed to meningitis, an epidural bleed, a subdural bleed, a subarachnoid bleed, and certain brain tumors. This finding also explains why this therapeutic approach for anti-CGRP active agents may not be effective for every headache patient.

As used herein, “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)2, Fv), single chain (ScFv), mutants thereof, fusion proteins comprising an antibody portion (such as domain antibodies), and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgGI, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

As used herein, “monoclonal antibody” or “mAb” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature, 256:495, or may be made by recombinant DNA methods such as described in U.S. Pat. No. 4,816,567. The monoclonal antibodies may also be isolated from phage libraries generated using the techniques described in McCafferty et al., 1990, Nature, 348:552-554, for example.

As used herein, “humanized” antibodies refer to forms of non-human (e.g., murine) antibodies that are specific chimeric immunoglobulins, immunoglobulin chains, or fragments thereof (such as Fv, Fab, Fab′, F(ab′)2 or other antigen-binding subsequences of antibodies) that contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and, biological activity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody.

As used herein, “human antibody” means an antibody having an amino acid sequence corresponding to that of an antibody produced by a human and/or has been made using any of the techniques for making human antibodies known in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nat. BiotechnoL, 14:309-314; Sheets et al., 1998, PNAS, (USA) 95:6157-6162; Hoogenboom and Winter, 1991, J. Mol. Biol, 227: 381; Marks et al., 1991, J. Mol. Biol, 222: 581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625, 126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., 1991, J. Immunol, 147 (I): 86-95; and U.S. Pat. No. 5,750,373.

As used herein, the terms “calcitonin gene-related peptide” and “CGRP”, which are used interchangeably, refer to any form of calcitonin gene-related peptide and variants thereof that retain at least part of the activity of CGRP. For example, CGRP may be α-CGRP or β-CGRP. As used herein, CGRP includes all mammalian species of native sequence CGRP, e.g., human, canine, feline, equine, and bovine.

As used herein, an “anti-CGRP antibody” refers to an antibody that modulates CGRP biological activity, or the CGRP pathway, including downstream pathways mediated by CGRP signaling, such as receptor binding and/or elicitation of a cellular response to CGRP. For example, an anti-CGRP antibody may block, inhibit, suppress or reduce the calcitonin gene related peptide (CGRP) pathway. The term anti-CGRP antibody encompasses both “anti-CGRP antagonist antibodies” and “anti-CGRP receptor antibodies.” In some embodiments, the anti-CGRP antibody is a monoclonal antibody (i.e., an anti-CGRP monoclonal antibody).

An “anti-CGRP antagonist antibody” refers to an antibody that is able to bind to CGRP and thereby inhibit CGRP biological activity and/or downstream pathway(s) mediated by CGRP signaling. An anti-CGRP antagonist antibody encompasses antibodies that modulate, block, antagonize, suppress or reduce CGRP biological activity, or otherwise antagonize the CGRP pathway, including downstream pathways mediated by CGRP signaling, such as receptor binding and/or elicitation of a cellular response to CGRP. In some embodiments, an anti-CGRP antagonist antibody binds CGRP and prevents CGRP binding to a CGRP receptor. In other embodiments, an anti-CGRP antagonist antibody binds CGRP and prevents activation of a CGRP receptor. Examples of anti-CGRP antagonist antibodies are provided herein.

An “anti-CGRP receptor antibody” refers to an antibody that is able to bind to a CGRP receptor and thereby modulate the CGRP pathway. Examples of anti-CGRP receptor antibodies are provided herein (e.g., erenumab).

A “gepant” refers to a small molecule CGRP antagonist. Examples of gepants are provided herein and include rimegepant, ubrogepant, vazegepant, atogepant, olcegepant, telcagepant, BI 44370 and MK-3207, and pharmaceutically acceptable salts thereof.

An “anti-CGRP active agent” refers to an active agent selected from the group consisting of anti-CGRP antibodies and gepants.

As used herein, the terms “GI,” “antibody GI,” “TEV-48125,” and “fremanezumab” are used interchangeably to refer to an anti-CGRP antagonist antibody produced by expression vectors having deposit numbers of ATCC PTA-6867 and ATCC PTA-6866. The characterization and processes for making antibody GI (and variants thereof) are described in PCT Publication No. WO2007/054809 and WHO Drug Information 30(2): 280-1 (2016), which are hereby incorporated by reference in its entirety.

The terms “ALD403,” and “eptinezumab” refer to an anti-CGRP antagonist antibody, which is a humanized IgGI monoclonal antibody from a rabbit precursor. Characterization and processes for making eptinezumab can be found in U.S. Publication No. U52012/0294797 and WHO Drug Information 30(2): 274-5 (2016), which are incorporated by reference in its entirety.

The terms “LY2951742,” and “galcanezumab” refer to an anti-CGRP antagonist antibody, which is a humanized IgG4 monoclonal antibody from a murine precursor. Characterization and processes for making galcanezumab can be found in U.S. Publication No. US2011/030571 1 and WHO Drug Information 29(4): 526-7 (2015), which are incorporated by reference in its entirety. Dosing and formulations associated with galcanezumab can be found in PCT Publication No. WO 2016/205037, which is also incorporated by reference in its entirety.

The terms “AMG334,” and “erenumab” refer to an anti-CGRP receptor antibody, which is a fully humanized IgG2 antibody. Characterization and processes for making erenumab can be found in U.S. Publication No. U52010/0172895, U.S. Pat. No. 9,102,731, and WHO Drug Information 30(2): 275-6 (2016), each of which are incorporated by reference in their entireties. Dosing and formulations associated with erenumab can be found in PCT Publication No. WO 2016/171742, which is also incorporated by reference in its entirety.

The term “rimegepant” refers to a specific small molecule CGRP antagonist and pharmaceutically acceptable salts thereof, the characterization of which and processes for making can be found in U.S. Pat. Nos. 8,314,117 and 8,759,372, each of which are incorporated by reference in its entirety.

The term “ubrogepant” refers to a specific small molecule CGRP antagonist and pharmaceutically acceptable salts thereof, the characterization of which and processes for making can be found in U.S. Pat. Nos. 8,754,096, 8,912,210 and 9,499,545, each of which is incorporated by reference in its entirety.

The term “vazegepant” refers to a specific small molecule CGRP antagonist and pharmaceutically acceptable salts thereof, the characterization of which and processes for making can be found in PCT Publication No. WO2011/123232, which is incorporated by reference in its entirety.

The term “atogepant” refers to a specific small molecule CGRP antagonist and pharmaceutically acceptable salts thereof, the characterization of which and processes for making can be found in U.S. Pat. No. 8,754,096 which is incorporated by reference in its entirety.

The term “olcegepant” refers to a specific small molecule CGRP antagonist and pharmaceutically acceptable salts thereof, the characterization of which and processes for making can be found in U.S. Pat. No. 6,344,449 which is incorporated by reference in its entirety.

The term “telcagepant” refers to a specific small molecule CGRP antagonist and pharmaceutically acceptable salts thereof, the characterization of which and processes for making can be found in U.S. Pat. No. 6,953,790 which is incorporated by reference in its entirety.

The term “BI 44370” refers to a specific small molecule CGRP antagonist and pharmaceutically acceptable salts thereof, the characterization of which and processes for making can be found in PCT Publication No. WO2005/092880 which is incorporated by reference in its entirety.

The term “MK-3207” refers to a specific small molecule CGRP antagonist and pharmaceutically acceptable salts thereof, the characterization of which and processes for making can be found in US Patent Publication No. U52007/0265225 which is incorporated by reference in its entirety.

The terms “polypeptide,” “oligopeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps,” substitution of one or more of the naturally occurring nucleotides with an analog, intemucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylates, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-0-allyl, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(0)S(“thioate”), P(S)S (“dithioate”), (0)NR2 (“amidate”), P(O)R, P(0)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

Diagnosis or assessment of headache is well-established in the art. References such as the International Classification of Headache Disorders, 3 rd edition (ICHD-III beta version; Cephalalgia (2013) 33(9): 629-808) can be used by a skilled practitioner to assess the type of headache experienced by a patient. Headaches within the scope of the instant invention include headaches of intracranial origin. Non-limiting examples of headaches of intracranial origin include migraine (e.g., chronic and episodic) and headache attributed to meningitis, an epidural bleed, a subdural bleed, a sub-arachnoid bleed, and certain brain tumors (wherein headache results from increased pressure in the skull).

For example, “chronic migraine” refers to headache occurring on 15 or more days per month for more than three months, which has the features of migraine headache on at least 8 days per month, whereas “episodic migraine” refers to headache occurring less than 15 days per month, and “high frequency episodic migraine” refers to headache occurring between 8 and 14 days per month. Diagnostic criteria for chronic migraine according to ICHD-III beta version, 2013 is as follows: A. Headache (tension-type-like and/or migraine-like) on >15 days per month for >3 months and fulfilling criteria B and C (below). B. Occurring in a patient who has had at least five attacks fulfilling certain criteria for migraine without aura and/or certain criteria for migraine with aura. C. On >8 days per month for >3 months, fulfilling any of the following: 1. certain criteria for migraine without aura; 2. certain criteria for migraine with aura; 3. believed by the patient to be migraine at onset and relieved by a triptan or ergot derivative, D. Not better accounted for by another headache diagnosis.

Skilled practitioners will be readily able to recognize a subject with any of the types of migraine headache described herein. Assessment may be performed based on subjective measures, such as patient characterization of symptoms. For example, migraine may be diagnosed based on the following criteria: 1) episodic attacks of headache lasting 4 to 72 hours; 2) with two of the following symptoms: unilateral pain, throbbing, aggravation on movement, and pain of moderate or severe intensity; and 3) one of the following symptoms: nausea or vomiting, and photophobia or phonophobia (Goadsby et al, N. Engl. J. Med. 346:257-270 2002). In some embodiments, assessment of headache (e.g., migraine) may be via headache hours, as described elsewhere herein. For example, assessment of headache (e.g., migraine) may be in terms of daily headache hours, weekly headache hours, monthly headache hours and/or yearly headache hours. In some cases, headache hours may be as reported by the subject.

As used herein, “treatment” is an approach for obtaining beneficial or desired clinical results. For purposes of this invention, beneficial or desired clinical results include, but are not limited to, one or more of the following: improvement in any aspect of headache, including lessening severity, alleviation of pain intensity, and other associated symptoms, reducing frequency of recurrence, reducing frequency of headache, increasing the quality of life of those suffering from the headache, and decreasing dose of other medications required to treat the headache. Using migraine as an example, other associated symptoms include, but are not limited to, nausea, vomiting, and sensitivity to light, sound, and/or movement. The terms “patient” and “subject” are used interchangeably herein. In some embodiments, the patient is a human.

As used herein, “acute treatment” is an approach for obtaining immediate beneficial or desired clinical results. For purposes of this invention, immediate beneficial or desired clinical results include, but are not limited to, one or more of the following: an increase in pain freedom and most bothersome symptom (MBS) freedom at two hours after dosing, wherein pain freedom can be defined as a reduction of moderate or severe headache pain to no headache pain and MBS freedom as the absence of the self-identified MBS, such as photophobia, phonophobia or nausea, an increase in pain relief at 2 hours, wherein pain relied can be defined as the reduction in migraine pain from moderate or severe severity to mild or none, an increase in sustained pain freedom at 2-48 hours, a reduction in the use of rescue medication within 24 hours, and an increase in the percentage of patients reporting normal function at two hours after dosing.

As used herein, “preventive treatment” is an approach for obtaining beneficial or desired clinical results over time. For purposes of this invention, beneficial or desired clinical results over time include, but are not limited to, one or more of the following: an improvement in aspects of headache, including reducing frequency of recurrence, reducing frequency of headache, increasing the quality of life of those suffering from the headache, and decreasing dose of other medications required to treat the headache.

As used herein, “preventing” is an approach to stop headache from occurring or existing in a subject, who is susceptible to the development of headache. For example, the patient may been previously diagnosed with chronic or episodic migraine. In other examples, the patient may have been diagnosed with meningitis, an epidural bleed, a subdural bleed, a sub-arachnoid bleed, or a brain tumor.

“Reducing headache incidence” or “reducing headache frequency” means any of reducing severity (which can include reducing need for and/or amount of (e.g., exposure to) other drugs and/or therapies generally used for this headache condition), duration, and/or frequency (including, for example, delaying or increasing time to next headache attack in an individual). As is understood by those skilled in the art, individuals may vary in terms of their response to treatment, and, as such, for example, a “method of reducing frequency of headache in an individual” reflects administering the anti-CGRP active agent based on a reasonable expectation that such administration may likely cause such a reduction in headache incidence in that particular individual.

“Ameliorating” headache or one or more symptoms of headache means a lessening or improvement of one or more symptoms of headache as compared to not administering an anti-CGRP active agent. “Ameliorating” also includes shortening or reduction in duration of a symptom.

As used herein, “controlling headache” refers to maintaining or reducing severity or duration of one or more symptoms of headache or frequency of headache (e.g., migraine) attacks in an individual (as compared to the level before treatment). For example, the duration or severity of head pain, or frequency of attacks is reduced by at least about any of 10%, 20%, 30%, 40%, 50%, 60%, 70%, 80%, 90% or more, in the individual as compared to the duration or severity of head pain, or frequency of attacks before treatment.

As used herein, a “headache hour” refers to an hour during which a subject experiences headache. Headache hours can be expressed in terms of whole hours (e.g., one headache hour, two headache hours, three headache hours, etc.) or in terms of whole and partial hours (e.g., 0.5 headache hours, 1.2 headache hours, 2.67 headache hours, etc.). One or more headache hours may be described with respect to a particular time interval. For example, “daily headache hours” may refer to the number of headache hours a subject experiences within a day interval (e.g., a 24-hour period). In another example, “weekly headache hours” may refer to the number of headache hours a subject experiences within a week interval (e.g., a 7-day period). As can be appreciated, a week interval may or may not correspond to a calendar week. In another example, “monthly headache hours” may refer to the number of headache hours a subject experiences within a month interval. As can be appreciated, a month interval (e.g., a period of 28, 29, 30, or 31 days) may vary in terms of number of days depending upon the particular month and may or may not correspond to a calendar month. In yet another example, “yearly headache hours” may refer to the number of headache hours a subject experiences within a year interval. As can be appreciated, a year interval (e.g., a period of 365 or 366 days) may vary in terms of number of days depending upon the particular year and may or may not correspond to a calendar year.

As used herein, a “headache day” refers to a day during which a subject experiences headache. Headache days can be expressed in terms of whole days (e.g., one headache day, two headache days, three headache days, etc.) or in terms of whole and partial days (e.g., 0.5 headache days, 1.2 headache days, 2.67 headache days, etc.). One or more headache days may be described with respect to a particular time interval. For example, “weekly headache days” may refer to the number of headache days a subject experiences within a week interval (e.g., a 7-day period). As can be appreciated, a week interval may or may not correspond to a calendar week. In another example, “monthly headache days” may refer to the number of headache days a subject experiences within a month interval. As can be appreciated, a month interval (e.g., a period of 28, 29, 30, or 31 days) may vary in terms of number of days depending upon the particular month and may or may not correspond to a calendar month. In yet another example, “yearly headache days” may refer to the number of headache days a subject experiences within a year interval. As can be appreciated, a year interval (e.g., a period of 365 or 366 days) may vary in terms of number of days depending upon the particular year and may or may not correspond to a calendar year.

As used therein, “delaying” the development of headache means to defer, hinder, slow, retard, stabilize, and/or postpone progression of the disease. This delay can be of varying lengths of time, depending on the history of the disease and/or individuals being treated. As is evident to one skilled in the art, a sufficient or significant delay can, in effect, encompass prevention, in that the individual does not develop headache. A method that “delays” development of the symptom is a method that reduces probability of developing the symptom in a given time frame and/or reduces extent of the symptoms in a given time frame, when compared to not using the method. Such comparisons are typically based on clinical studies, using a statistically significant number of subjects.

“Development” or “progression” of headache means initial manifestations and/or ensuing progression of the disorder. Development of headache can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of headache includes initial onset and/or recurrence.

Migraine can be defined by both its periodicity and its specific phases. As used herein, the “inter-ictal phase” of a migraine refers to the interval between two migraine attacks, the “pre-ictal phase” refers to the time before the headache starts, when the patient may develop premonitory symptoms, including appetite changes, thirst, yawning, or others, the “ictal phase” refers to the time period when the patient experiences headache and which last for between 4-72 hours, and the “post-ictal phase” refers to the time within the inter-ictal phase following the cessation of the headache and typically characterized by non-headache symptoms such as cognitive deficits, fatigue, and others.

“Responder rate” means the proportion of patients reaching at least a 50% reduction in monthly average number of migraine days during a predetermined treatment period. In one embodiment of the invention, the predetermined treatment period is 3 months. In another embodiment of the invention, the predetermined treatment period is 6 months. In yet another embodiment of the invention, the predetermined treatment period is 12 months.

Migraine can be defined by both its periodicity and its specific phases. As used herein, the “inter-ictal phase” of a migraine refers to the interval between two migraine attacks, the “pre-ictal phase” refers to the time before the headache starts, when the patient may develop premonitory symptoms, including appetite changes, thirst, yawning, or others, the “ictal phase” refers to the time period when the patient experiences headache and which last for between 4-72 hours, and the “post-ictal phase” refers to the time within the inter-ictal phase following the cessation of the headache and typically characterized by non-headache symptoms such as cognitive deficits, fatigue, and others.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as reducing pain intensity, duration, or frequency of headache attack, and decreasing one or more symptoms resulting from headache (biochemical, histological and/or behavioral), including its complications and intermediate pathological phenotypes presenting during development of the disease, increasing the quality of life of those suffering from the disease, decreasing the dose of other medications required to treat the disease, enhancing effect of another medication, and/or delaying the progression of the disease of patients. An effective dosage can be administered in one or more administrations. For purposes of this disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, “allodynia” refers to pain experienced by a patient and due to a stimulus that does not normally elicit pain (International Association for the Study of Pain, 2014-2015, “Allodynia and Hyperalgesia in Neuropathic Pain”).

As used herein, “hyperalgesia” refers to an increase in pain experienced by a patient from a stimulus that normally provokes pain (International Association for the Study of Pain, 2014-2015, “Allodynia and Hyperalgesia in Neuropathic Pain”).

Both allodynia and hyperalgesia can be distinguished and quantified by one of skill in the art by methods such as, for example, quantitative sensory testing (QST) (Rolke (2006) et al. Pain 123: 231-243). Rolke et al. teaches QST reference data for obtaining the full somatosensory phenotype of a patient, in both relative and absolute terms. For example, Rolke et al. describes a test for mechanical pain sensitivity (MPS) as a means for detecting pinprick hyperalgesia. In such a test, MPS can be assessed using a set of pinprick stimuli to obtain a stimulus-response function for pinprick-evoked pain (where the strongest pinprick force is about eight-times the mean mechanical pain threshold). Subjects can be asked to give the pain a rating for each stimulus on a ‘0-100’ scale, wherein ‘0’ indicates no pain and ‘ 100’ indicates highest pain. A certain number of pinpricks are delivered to the subject at certain time intervals to avoid wind-up. After each pinprick, the subject provides numerical pain ratings. MPS is then calculated as the geometric mean (compound measure) of all numerical ratings for pinprick stimuli (Rolke et al. at p. 233).

As used herein, “sensitization” is the process whereby the strength of the stimulus that is needed to generate a response decrease over time, while the amplitude of the response increases.

The phrase “headache primarily experienced in a portion of the head” refers to description by the patient of having headache (experienced as, e.g., pain) in an identified part of the head. Examples of “portions of the head” include one-side periorbital, one-side temporal, one eye, a small area in the back of the head (e.g., just lateral to the midline), a small area on the top of the head, a small area in the middle of the forehead, a ‘dot’ (e.g., 10×10 mm) where the supraorbital nerve exits the skull (i.e., in the medial end of the eyebrow) and a small area across the forehead. One of skill in the art would be able to assess whether a patient is experiencing headache in a portion of the head based on the patient's description (Noseda, R. et al. (2016) Brain. 139 (7): 1971-1986).

The majority of episodic migraineurs seeking secondary or tertiary medical care exhibit signs of allodynia and/or hyperalgesia during the ictal phase of migraine, but not during the inter-ictal phase (Burstein et al. 2000b; Lipton et al. 2008; Bigal et al. 2008; Burstein et al. 2000a). In contrast, chronic migraine patients commonly exhibit sign of allodynia and/or hyperalgesia both during acute migraine attacks as well as during the inter-ictal phase. Mechanistically, allodynia is thought to be mediated by sensitization of central trigeminovascular neurons in the spinal trigeminal nucleus (Burstein et al. 1998). Furthermore, the presence of inter-ictal allodynia and/or hyperalgesia is mediated by central trigeminovascular neurons whose sensitization state does not depend on incoming pain signals from the meninges, whereas the absence of inter-ictal allodynia and/or hyperalgesia in migraine patients is explained by the existence of central trigeminovascular neurons whose sensitized state depends on pain signals that come from the periphery.

Gepants are unlikely to cross the blood-brain-barrier and inhibit the central trigeminovascular neurons directly. The inventors of the present application have determined that the therapeutic ability of gepants dictate that in some episodic, most likely high frequency, and chronic migraine patients, central sensitization and allodynia and/or hyperalgesia remain dependent of pain signals that originate in the meninges and that patients who will respond to these agents will be those in which the ongoing peripheral input is required to maintain the central sensitization, whereas the non-responders will be those in which the ongoing peripheral input is not required to maintain the central sensitization. Therefore, the peripheral site of action of gepants will allow these medications to provide acute and preventive treatment for both episodic and chronic migraine patients whose state of central sensitization depends on pain signals that arrive from the meninges but not in those patients in which the state of central sensitization is independent of the pain signals that arrive from the meninges. Such patients may present as not exhibiting allodynia and/or hyperalgesia in either the ictal and/or inter-ictal phase of the migraine.

Provided herein is a method for reducing headache (e.g., migraine) frequency in a patient. The method includes determining whether the patient exhibits allodynia and/or hyperalgesia during the interictal phase of a migraine and administering to the patient that does not exhibit signs of allodynia and/or hyperalgesia during the interictal phase of the migraine a gepant. In one embodiment of the invention, the treatment is preventive. In another embodiment of the invention, the treatment is acute.

Also provided herein is a method of treating migraine in a patient. The method includes determining whether the patient exhibits allodynia and/or hyperalgesia during the interictal phase of a migraine and administering to the patient that does not exhibit signs of allodynia and/or hyperalgesia during the interictal phase of the migraine a gepant. In one embodiment of the invention, the treatment is preventive. In another embodiment of the invention, the treatment is acute.

Also provided herein is a method of for reducing headache (e.g., migraine) frequency in a patient suffering from migraines. The method can include determining whether the patient exhibits, or does not exhibit, allodynia and/or hyperalgesia during an interictal phase of a migraine and administering to the patient that does not exhibit signs of allodynia and/or hyperalgesia during the interictal phase of the migraine a gepant. The method can also include determining whether the patient exhibits, or does not exhibit, allodynia and/or hyperalgesia during an interictal phase of a migraine and administering to the patient that does not exhibit signs of allodynia and/or hyperalgesia during the ictal phase of the migraine a gepant. The method can also include determining whether the patient exhibits, or does not exhibit, allodynia and/or hyperalgesia during the ictal phase of a migraine and administering to the patient that does not exhibit signs of allodynia and/or hyperalgesia during the ictal phase of the migraine a gepant.

Also provided herein is a method for reducing headache (e.g., migraine) frequency in a patient. The method includes determining whether the patient exhibits allodynia and/or hyperalgesia during the interictal phase of a migraine and administering to the patient that does not exhibit signs of allodynia and/or hyperalgesia during the ictal phase of the migraine a gepant. In one embodiment of the invention, the treatment is preventive. In another embodiment of the invention, the treatment is acute.

Also provided herein is a method of treating migraine in a patient. The method includes determining whether the patient exhibits allodynia and/or hyperalgesia during the interictal phase of a migraine and administering to the patient that does not exhibit signs of allodynia and/or hyperalgesia during the ictal phase of the migraine a gepant. In one embodiment of the invention, the treatment is preventive. In another embodiment of the invention, the treatment is acute.

Also provided herein is a method for reducing headache (e.g., migraine) frequency in a patient. The method includes determining whether the patient exhibits allodynia and/or hyperalgesia during the ictal phase of a migraine and administering to the patient that does not exhibit signs of allodynia and/or hyperalgesia during the ictal phase of the migraine a gepant. In one embodiment of the invention, the treatment is preventive. In another embodiment of the invention, the treatment is acute.

Also provided herein is a method of treating migraine in a patient. The method includes determining whether the patient exhibits allodynia and/or hyperalgesia during the ictal phase of a migraine and administering to the patient that does not exhibit signs of allodynia and/or hyperalgesia during the ictal phase of the migraine a gepant. In one embodiment of the invention, the treatment is preventive. In another embodiment of the invention, the treatment is acute.

In one embodiment of the invention, the gepant is administered within 3 hours of the start of the ictal phase of the migraine. In another embodiment of the invention, the gepant is administered within 150 minutes of the start of the ictal phase of the migraine. In another embodiment of the invention, the gepant is administered within 120 minutes of the start of the ictal phase of the migraine. In another embodiment of the invention, the gepant is administered within 105 minutes of the start of the ictal phase of the migraine. In another embodiment of the invention, the gepant is administered within 90 minutes of the start of the ictal phase of the migraine. In another embodiment of the invention, the gepant is administered within 75 minutes of the start of the ictal phase of the migraine. In another embodiment of the invention, the gepant is administered within 60 minutes of the start of the ictal phase of the migraine. In another embodiment of the invention, the gepant is administered within 45 minutes of the start of the ictal phase of the migraine. In another embodiment of the invention, the gepant is administered within 30 minutes of the start of the ictal phase of the migraine. In yet another embodiment of the invention, the gepant is administered within 15 minutes of the start of the ictal phase of the migraine.

In one embodiment of the invention, the gepant is administered ictally before the patient is centrally sensitized. In another embodiment of the invention, the gepant is administered ictally before the patient develops ictal allodynia and/or hyperalgesia.

In one embodiment of the invention, the subject suffers from episodic migraine. In one embodiment of the invention, the subject suffers from high frequency episodic migraine. In another embodiment of the invention, the subject suffers from chronic migraine.

In one embodiment of the invention, the determination of whether the subject exhibits allodynia and/or hyperalgesia is by quantitative sensory testing (QST). In another embodiment of the invention, the determination of whether the subject exhibits allodynia and/or hyperalgesia is by questionnaire. In another embodiment of the invention, the determination of whether the subject exhibits allodynia and/or hyperalgesia is by both quantitative sensory testing (QST) and questionnaire. In one embodiment of the invention, the QST and/or questionnaire are determined at a healthcare facility. In another embodiment of the invention, the QST and/or questionnaire are determined at the subject's place of residence.

Quantitative Sensory testing (QST) should be performed preferably in a quiet room away from noise and distraction. There, patients should be allowed to choose their most comfortable position (sitting on a chair or lying in bed) during the sensory testing. In each testing session, pain thresholds to hot and mechanical stimulation are determined in the skin over the site to where the pain is referred to, with the periorbital and temporal regions being the most common sites tested. Heat skin stimuli should be delivered through a 30×30 mm² thermode (Q-Sense 2016, Medoc) attached to the skin at a constant pressure and the participant's pain thresholds determined by using the Method of Limit analysis.

Allodynia testing should be performed to determine pain thresholds with the skin allowed to adapt to a temperature of 32° C. for 5 minutes and then warmed up at a slow rate (1° C./sec) until pain sensation is perceived, at which moment the subject will be allowed to stop the stimulus by pressing a button on a patient response unit. Heat stimuli should be repeated three times each and the mean of recorded temperatures will be considered threshold. Pain threshold to mechanical stimuli can be determined by using a set of up to 20 calibrated von Frey hairs (VFH, Stoelting). Each VFH monofilament is assigned a scalar number in an ascending order and each monofilament should be applied to the skin 3 times (for 2 sec). The smallest VFH number capable of inducing pain at two out of three trials will be considered as threshold. Skin sensitivity can also be determined by recording the subject's perception of soft skin brushing, which is a dynamic mechanical stimulus, as distinguished from the VFH, which is a static mechanical stimulus.

Hyperalgesia testing should be performed to determine when a painful stimulus is perceived as more painful than usual. 3 supra-threshold heat and mechanical stimuli should be applied to the skin. The value of the supra-threshold stimulus can be determined during the allodynia testing which the subject will have already undertaken. In this test, the skin should be exposed to 3 supra-threshold stimuli (1 above-threshold), each lasting 10 seconds and separated by 10 seconds (i.e., inter-stimulus interval of 10 seconds). At the end of each stimulus, the patient should have 10 seconds to identify the intensity of the pain using a visual analog scale (VAS) of 0-10 (o=no pain, 10=most imaginable pain). A similar test can be administered using supra-threshold mechanical stimulation.

In one embodiment of the invention, the determination of whether the subject exhibits allodynia and/or hyperalgesia is by a determination of whether the subject has a heat pain threshold of below 41° C. and/or a cold pain threshold of above 21° C. and/or a mechanical pain threshold of below 30 g for skin indentation with calibrated von Frey hairs.

In one embodiment of the invention, the subject is determined to exhibit allodynia and/or hyperalgesia by exhibiting a heat pain threshold of below 41° C. and/or a cold pain threshold of above 21° C. and/or a mechanical pain threshold of below 30 g for skin indentation with calibrated von Frey hairs.

In one embodiment of the invention, the subject is determined to not exhibit allodynia and/or hyperalgesia by exhibiting a heat pain threshold of above 40° C. and/or a cold pain threshold of above 20° C. and/or a mechanical pain threshold of above 30 g for skin indentation with calibrated von Frey hairs.

In one embodiment of the invention, the questionnaire is specifically designed to capture the presence or absence of inter-ictal allodynia and/or hyperalgesia. In another embodiment of the invention, the questionnaire is specifically designed to capture the presence or absence of ictal allodynia and/or hyperalgesia, such as the Allodynia Symptom Checklist (ASC-12) (Lipton R B et al 2008). In one embodiment of the invention, the questionnaire is incorporated as part of an e-diary. In one embodiment of the invention, the e-diary is recorded daily by the subject over a time period of at least seven days beginning at least twenty-four hours into the post-ictal phase of the migraine.

A specifically designed questionnaire for identifying inter-ictal allodynia and/or hyperalgesia could be a variation of the Allodynia Symptom Checklist (ASC-12) (Lipton R B et al 2008) which has been modified for the inter-ictal phase of a migraine rather than for the ictal phase where the ASC-12 is typically used. Such modifications might result in the removal of questions relating to wearing necklaces or contact lenses and the scaling might be ranked similarly to the ASC-12 or it might be a more simplified ranking of never/rarely (score=0) and at least some of the time (score=0). With such modifications, a finding of no allodynia might relate to a score of 0, of 1, of 2, of 3, of 4 or of 5.

In one embodiment of the invention, the determination of the absence of allodynia and/or hyperalgesia by the review of the questionnaire by a suitably qualified healthcare professional. In one embodiment of the invention, the determination of the absence of allodynia and/or hyperalgesia is by a questionnaire score of no more than 5. In one embodiment of the invention, the determination of the absence of allodynia and/or hyperalgesia is by a questionnaire score of no more than 5, no more than 4, no more than 3, no more than 2, no more than 1 or a questionnaire score of 0.

In one embodiment of the invention, the preventative treatment comprises a reduction in responder rate, i.e., a reduction in the proportion of patients reaching at least a 50% reduction in monthly average number of migraine days during the treatment period. In another embodiment of the invention, the preventive treatment comprises a reduction in the number of monthly migraine headache days over a treatment period of at least three months. In another embodiment of the invention, the preventive treatment comprises a reduction in the use of acute headache medication. In another embodiment of the invention, the preventive treatment comprises an improvement in the subject's functionality. In another embodiment of the invention, wherein the preventive treatment comprises an improvement in the subject's Quality of Life (QoL). In another embodiment of the invention, the preventive treatment comprises an improvement in the subject's headache severity. In another embodiment of the invention, the preventive treatment comprises a reduction in the number of monthly non-migraine headache days over a treatment period of at least three months. In yet another embodiment of the invention, the preventive treatment comprises a reduction in the subject's photophobia, phonophobia and/or nausea.

In one embodiment of the invention, the acute treatment comprises an increase in pain freedom. In another embodiment of the invention, the acute treatment comprises an increase in most bothersome symptom freedom. In another embodiment of the invention, the acute treatment comprises an increase in pain freedom and most bothersome symptom freedom. In another embodiment of the invention, the acute treatment comprises an increase in pain relief. In another embodiment of the invention, the acute treatment comprises an increase in sustained pain freedom. In another embodiment of the invention, the acute treatment comprises a decrease in use of rescue medication. In yet another embodiment of the invention, the acute treatment comprises an increase in normal functioning. In one embodiment of the invention, the subject administered a gepant remains free of allodynia and/or hyperalgesia for at least three months after initiation of the treatment. In one embodiment of the invention, the subject administered a gepant remains free of allodynia and/or hyperalgesia for at least three months, at least four months, at least five months, at least six months, at least seven months, at least eight months, at least nine months, at least ten months, at least eleven months, or at least twelve months after initiation of the treatment.

In one embodiment of the invention, the gepant is administered while the subject is migraine free.

Selecting the patient includes determining whether the patient's headache is mediated by HT neurons. Skilled practitioners will appreciate that such a determination can be made in any number of ways described herein, such as by observation of HT neuron activity and/or administering a monoclonal antibody that modulates the CGRP pathway to the patient and determining whether the antibody reduces hyperalgesia (as measured, for example, by QST), and/or determining that the patient's headache pain is localized (e.g., experienced most intensely or primarily) in a portion of the head.

Example 1 describes the means by which neurons could be identified and selected (HT v. WDR neurons) in a rat. This example further describes the observations made in connection with the activation and sensitization of each of these types of neurons after induction of CSD.

Patients who experience hyperalgesia, wherein the hyperalgesia is reduced (e.g., reversed or eliminated) upon administration of a monoclonal antibody that modulates (e.g., blocks, inhibits, suppresses or reduces) the CGRP pathway, are likely to respond to a course of treatment comprising an anti-CGRP active agent that modulates (e.g., blocks, inhibits, suppresses or reduces) the CGRP pathway, e.g., a longer course and/or higher dose course of treatment with an anti-CGRP active agent. If the anti-CGRP active agent reduces the headache in hyperalgesic patients, it confirms that the headache was mediated by the HT neurons because the anti-CGRP active agent does not inhibit the other class of nociceptive neurons, the WDR, as shown in Example 1. Example 2 describes the experimental design of QST that is useful in determining whether a patient experiences allodynia and/or hyperalgesia, and whether it is reduced upon treatment with a gepant.

Likewise, a patient who experiences allodynia, wherein the allodynia is reduced (e.g., reversed or eliminated) upon administration of a gepant that antagonises the CGRP pathway, is likely to respond to a course of treatment comprising a gepant that antagonises the CGRP pathway, e.g., a longer course and/or higher dose course of treatment with an anti-CGRP active agent.

Thus, a patient that responds to treatment with a gepant may experience a reduction, reversal, or elimination of both hyperalgesia and allodynia after a first course of treatment.

Further, a patient who experiences does not allodynia and/or hyperalgesia when headache-free, i.e., during the interictal phase of the migraine may be treated by administration of a gepant that antagonises the CGRP pathway. Identification of this patient population should allow for improved responder rates to gepant and other anti-CGRP active agent therapies.

Further, it is known that high-threshold neurons exhibit small receptive fields, while wide dynamic range neurons exhibit large receptive fields. Thus, headache pain localized (or primarily experienced) in a portion of the head may identify a patient who will respond favorably to treatment with a gepant that antagonises the CGRP pathway.

In another embodiment, the patient is or was previously diagnosed as having episodic or chronic migraine. In such a patient, the anti-CGRP active agent can be administered while the patient is free of migraine, or experiencing the early stages of migraine or mild migraine.

In another embodiment, the patient is or was previously diagnosed as having meningitis, an epidural bleed, a subdural bleed, a sub-arachnoid bleed, or a brain tumor. In these instances, the headache may be attributed to meningitis, an epidural bleed, a subdural bleed, a sub-arachnoid bleed, or a brain tumor.

Accordingly, in certain methods described herein, a gepant to be used in the methods described herein may be selected from the group consisting of rimegepant, ubrogepant, vazegepant, atogepant, olcegepant, telcagepant, BI 44370, MK-3207, and bioequivalents thereof and can be administered at a dose of from about 10 mg to about 250 mg, e.g., a dose of about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 150 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, or about 250 mg. Administration of the dose can be once daily, more than once daily, or intermittently within a week or longer.

Administration of a gepant can be by any means known in the art, including: orally, intravenously, subcutaneously, intraarterially, intramuscularly, intranasally (e.g., with or without inhalation), intracardially, intraspinally, intrathoracically, intraperitoneally, intraventricularly, sublingually, transdermally, and/or via inhalation.

Administration may be systemic, e.g., intravenously, or localized. In some embodiments, an initial dose and one or more additional doses are administered via same route, i.e., subcutaneously or intravenously. In some embodiments, the one or more additional doses are administered via a different route than the initial dose, i.e., the initial dose may be administered intravenously and the one or more additional doses may be administered subcutaneously.

In some instances, methods described herein can further include administering to the patient a second agent simultaneously or sequentially with the gepant. The second agent can be non-steroidal anti-inflammatory drugs (NSAID) and/or triptans and/or a 5 hydroxytryptamine IF receptor agonist (i.e., a serotonin receptor agonist). In some instances, the second agent is an agent that is administered to the patient prophylactically.

Non-limiting examples of NSAIDs that can be used in combination with an anti-CGRP antibody include aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin or zomepirac, cyclooxygenase-2 (COX-2) inhibitors, celecoxib, rofecoxib, meloxicam, JTE-522, L-745,337, NS398, or a pharmaceutically acceptable salt thereof. Non-limiting examples of triptans that can be used in combination with an anti-CGRP antibody include sumatriptan, zolmitriptan, naratriptan, rizatriptan, eletriptan, almotriptan, and afrovatriptan. A non-limiting example of a 5 hydroxytryptamine IF receptor agonist is Lasmiditan.

The preventing, treating, or reducing of the methods provided herein can comprise reducing the number of headache hours of any severity, reducing the number of migraine hours of any severity, reducing the number of monthly headache days of any severity, reducing the number of monthly migraine days of any severity, reducing the use of any acute headache medications, reducing a 6-item Headache Impact Test (HIT-6) disability score, improving 12-Item Short Form Health Survey (SF-12) score (Ware et al., Med. Care 4:220-233, 1996), reducing Patient Global Impression of Change (PGIC) score (Hurst et al, J. Manipulative Physiol. Ther. 27:26-35, 2004), improving Sport Concussion Assessment tool 3 (SCAT-3) score (McCrory et al. British J. Sport. Med. 47:263-266, 2013), or any combination thereof. In some embodiments, the number of monthly headache or migraine days can be reduced for at least seven days after a single administration.

In some embodiments, monthly headache or migraine hours experienced by the subject after said administering is reduced by 40 or more hours (e.g., 45, 50, 55, 60, 65, 70, 75, 80, or more) from a pre-administration level in the subject. Monthly headache or migraine hours may be reduced by more than 60 hours. In some embodiments, monthly headache or migraine hours experienced by the subject after said administering are reduced by 25% or more (e.g., 30%, 35%, 40%, 45%, 50%, or more) relative to a pre-administration level in the subject. Monthly headache or migraine hours may be reduced by 40% or more. In some embodiments, monthly headache or migraine days experienced by the subject after said administering is reduced by three or more days (e.g., 3, 4, 5, 6, 7, 8, 9, 10, 11, 12, 13, 14, 15, 16, 17, 18, 19, 20, or more days) from a pre-administration level in the subject. In some embodiments, the number of monthly headache or migraine days can be reduced by at least about 50% from a pre-administration level in the subject. Thus, in some aspects, the number of monthly headache or migraine days can be reduced by at least about 50%, at least about 55%, at least about 60%, at least about 65%, at least about 70%, at least about 75%, at least about 80%, or at least about 90%.

A gepant and compositions thereof provided herein can also be used in conjunction with other agents that serve to enhance and/or complement the effectiveness of the antibody.

Also provided herein are kits for use in the instant methods. Kits can include one or more containers comprising an antibody described herein (e.g., a gepant, and instructions for use in accordance with any of the methods described herein. Generally, these instructions comprise a description of administration of the antibody to select and treat a patient according to any of the methods described herein. For example, the kit may comprise a description of how to select a patient suitable for treatment based on identifying whether or not that patient exhibits allodynia and/or hyperalgesia during the interictal phase of their migraine. In still other embodiments, the instructions include a description of how to administer a gepant to the patient to reduce the frequency of headache.

Accordingly, a kit can include, e.g., a pre-filled syringe, pre-filled syringe with a needle safety device, injection pen, or auto-injector comprising a dose of a gepant; and instructions to determine whether a patient's allodynia and/or hyperalgesia occurs during the interictal phase of their migraine. Alternatively or in addition, the instructions may instruct to determine whether a patient exhibits allodynia and/or hyperalgesia, reducible by administering a gepant, and/or to determine whether a patient's headaches are primarily experienced in a portion of the head (e.g., one-side periorbital, one-side temporal, or one eye).

Another exemplary kit may comprise a gepant that antagonises the CGRP pathway and detailed instructions on how to administer QST to a patient or instructions on conducting a patient questionnaire and analyzing the responses to determine whether the patient's patient's allodynia and/or hyperalgesia occurs during the interictal phase of their migraine.

In addition to instructions relating to the identification of responders, the kits may further comprise instructions for further treatment with a gepant, including information relating to dosage, dosing schedule, and route of administration for the intended treatment (e.g., instructions to achieve reduction in headache frequency once a patient is identified as a responder according to the instructions of the kit).

In a kit provided herein, a gepant provided in a kit can include rimegepant, ubrogepant, vazegepant, atogepant, olcegepant, telcagepant, BI 44370, MK-3207 or a pharmaceutically acceptable salt thereof.

The kits of this invention can be provided in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a gepant. The container may further comprise a second pharmaceutically active agent. Kits may optionally provide additional components such as buffers and interpretive information. Normally, the kit comprises a container and a label or package insert(s) on or associated with the container.

The following Examples are provided to illustrate but not limit the invention. It is understood that the examples and embodiments described herein are for illustrative purposes only and that various modifications or changes in light thereof will be suggested to persons skilled in the art and are to be included within the spirit and purview of this application. All publications, patents, and patent applications cited herein are hereby incorporated by reference in their entirety for all purposes to the same extent as if each individual publication, patent or patent application were specifically and individually indicated to be so incorporated by reference.

### EXAMPLES

### Example 1: Selective Inhibition of Trigeminovascular Neurons by the Humanized Monoclonal Anti-CGRP Antibody (Fremanezumab. TEV-48125)

The purpose of this study was to better understand how the CGRP-mAb fremanezumab (TEV-48125) modulates meningeal sensory pathways. To answer this question single-unit recording was used to determine the effects of fremanezumab (30 mg/kg IV) and a IgG2 isotype control antibody (isotype-conAb) on spontaneous and evoked activity in naive and CSD-sensitized trigeminovascular neurons in the spinal trigeminal nucleus of anesthetized male and female rats. The study demonstrates that in both sexes fremanezumab inhibited naive high-threshold (HT) but not wide-dynamic range trigeminovascular neurons, and that the inhibitory effects on the neurons were limited to their activation from the intracranial dura but not facial skin or cornea. Additionally, when given sufficient time, fremanezumab prevents activation and sensitization of HT neurons by cortical spreading depression.

A. Materials and Methods

Surgical Preparation

Experiments were approved by the Beth Israel Deaconess Medical Center and Harvard Medical School standing committees on animal care, and in accordance with the U. S. National Institutes of Health Guide for the Care and Use of Laboratory Animals. Male and female Sprague-Dawley rats (250-350 g) were anesthetized with urethane (0.9-1.2 g/kg i.p.). They were fitted with an intra-tracheal tube to allow artificial ventilation (0.1 L/min of O₂), and an intra-femoral-vein cannula for later infusion of drugs. Rats were placed in a stereotaxic apparatus, and core temperature was kept at 37° C. using a heating blanket. End-tidal CC was continuously monitored and kept within physiological range (3.5-4.5 pCC). Once stabilized, rats were paralyzed with rocuronium bromide (10 mg/ml, 1 ml/hr continuous intravenous infusion) and ventilated. For stimulation of the cranial dura later in the experiment, a 5×5-mm opening was carefully carved in the parietal and occipital bones in front and behind the lambda suture, directly above the left transverse sinus. The exposed dura was kept moist using a modified synthetic interstitial fluid (135 mM NaCl, 5 mM KCl, 1 mM MgCh, 5 mM CaCh, 10 mM glucose and 10 mM Hepes, pH 7.2). For single-unit recording in the spinal trigeminal nucleus, a segment of the spinal cord between the obex and C2 was uncovered from overlying tissues, stripped of the dura mater, and kept moist with mineral oil.

Neuronal Identification and Selection

To record neuronal activity, a tungsten microelectrode (impedance 3-4 MO) was lowered repeatedly into the spinal trigeminal nucleus (STN) in search of central trigeminovascular neurons receiving convergent input from the dura and facial skin. Trigeminovascular neurons were first identified based on their responses to electrical stimulation of the dura. They were selected for the study if they exhibited discrete firing bouts in response to ipsilateral electrical (0.1-3.0 mA, 0.5 msec, 0.5 Hz pulses) and mechanical (with a calibrated von Frey monofilaments) stimulation of the exposed cranial dura and to mechanical stimulation of the facial skin and cornea. Dural receptive fields were mapped by indenting the dura (with the 4.19 g VFH monofilament) at points separated by 1 mm mediolaterally and rostrocaudally. Points at which dural indentation produced a response in >50% of the trials were considered inside the neurons receptive field. Cutaneous receptive fields were mapped by applying innocuous and noxious mechanical stimulation to all facial skin areas and the cornea. An area was considered outside the receptive field if no stimulus produced a response in >50% of the trials. Responses to mechanical stimulation of the skin were determined by applying brief (10 s) innocuous and noxious stimuli to the most sensitive portion of the cutaneous receptive field. Innocuous stimuli consisted of slowly passing a soft bristled brush across the cutaneous receptive field (one 5-s brush stroke from caudal to rostral and one 5-s brush stroke from rostral to caudal) and pressure applied with a loose arterial clip. Noxious stimuli consisted of pinch with a strong arterial clip (Palecek et al, 1992, J. Neurophysiol. 67: 1562-1573; Dado et al, 1994, J. Neurophysiol. 71:981-1002; Burstein et al., 1998, J. Neurophysiol. 79:964-982). More intense or prolonged stimuli were not used to avoid inducing prolonged changes in spontaneous neuronal discharge or response properties. Responses to mechanical stimulation of the cornea consisted of gentle and slow brushing strokes with a thin paintbrush (about 10 hair-follicles). Two classes of neurons were thus identified: wide-dynamic-range (WDR) neurons (incrementally responsive to brush, pressure and pinch), and high-threshold (HT) neurons (unresponsive to brush). Real-time waveform discriminator was used to create and store a template for the action potential evoked in the neuron under study by electrical pulses on the dura; spikes of activity matching the template waveform were acquired and analyzed online and offline using Spike 2 software (CED, Cambridge, UK). Induction and recording of cortical spreading depression.

Cortical spreading depression (CSD) was induced mechanically by inserting a glass micropipette (tip diameter 25 μm) about 1 mm into the visual cortex for 10 sec. At a propagation rate of 3-5 mm/min, a single wave of CSD was expected to enter the neuronal receptive field within 1-2 min of cortical stimulation. For verification of CSD, cortical activity was recorded (electrocorticogram) with a glass micropipette (0.9% saline, −1 megohm, 7 um tip) placed just below the surface of the cerebral cortex (approximately 100 μm). The electrocorticogram electrode was positioned about 6 mm anterior to the visual cortex. Treatment with the monoclonal anti-CGRP antibody fremanezumab (TEV-48125). Fremanezumab (also known as TEV-48125/LBR-101/RN-307) (TEVA Pharmaceutical Industries Ltd., Israel) is a humanized monoclonal anti-CGRP antibody (CGRP-mAb). It was diluted in saline to a final dose of 30 mg/kg and administered intravenously (bolus injection, total volume 0.6-0.7 ml). A corresponding human IgG2 isotype control antibody (isotype-conAb) was also diluted in saline to a final dose of 30 mg/kg and administered intravenously (bolus injection, total volume 1.6-2.0 ml).

### Experimental Protocol

The experimental protocol included two parts. The first part was designed to compare CGRP-mAb vs isotype-conAb effects on spontaneous and induced activity of naive trigeminovascular neurons, and the second part was designed to test CGRP-mAb vs isotype-conAb effects on the activation and sensitization of trigeminovascular neurons by CSD. Both parts included sampling of WDR and HT neurons in male and female rats. In the first part, the baseline neuronal profile was established by (a) mapping the dural, cutaneous and corneal receptive field; (b) measuring responses (mean spikes/sec) to mechanical stimulation of the dura (with a fixed force), skin (brush, pressure, pinch) and cornea (brush), and (c) measuring spontaneous firing rate (recorded over 30 min prior to treatment). Once the baseline was established, CGRP-mAb or isotype-conAb were administered and receptive fields were remapped, neuronal responses to stimulation of the dura, skin and cornea were re-examined, and the spontaneous activity rate was re-sampled at 1, 2, 3, and 4 hours post-treatment. The resulting values for each measure were then compared with the respective baseline values obtained before treatment. In the second part, CSD was induced 4 hours after administration of CGRP-mAb or isotype-conAb and 2 hours later (i.e., 6 hours after treatment) receptive field size, spontaneous activity rate, and response magnitude to stimulation of the dura, skin and cornea were measured again. The resulting post-CSD values for each measure were then compared with the respective pre-CSD values obtained at the 4-hour post-treatment time. This part was initiated only in cases in which the physiological condition of the rats (heart rate, blood pressure, respiration, end tidal C02) and the neuronal isolation signal (signal-to-noise ratio >1:3) were stable at the 4-hour post-treatment time point.

At the conclusion of each experiment, a small lesion was produced at the recording site (anodal DC of 15 μA for 15 sec) and its localization in the dorsal horn was determined postmortem using histological analysis as described elsewhere (Zhang et al. (2011) Ann. Neurol. 69: 855-865). Only one neuron was studied in each animal.

Data Analysis

To calculate the response magnitude to each stimulus, the mean firing frequency occurring before the onset of the first stimulus (30 min for spontaneous activity, 10 sec for mechanical stimulation of the dura, skin and cornea) was subtracted from the mean firing frequency that occurred throughout the duration of each stimulus. In the first part of the study, corresponding values for each measure (determined at 1, 2, 3, 4 hrs after treatment) were compared with the respective baseline values obtained before fremanezumab or isotype-conAb administration. In the second part of the study, resulting values for each measure (determined 2 hours after CSD induction) were compared with the respective values obtained before CSD induction in the 2 treatment groups (fremanezumab and isotype-conAb). A neuron was considered activated when its mean firing rate after CSD exceeded its mean baseline activity by 2 standard deviations of that mean fora period >10 min, which translated to >33% increase in activity. A neuron was considered sensitized if 2 hours after occurrence of CSD it exhibited enhanced responses to at least 3 of the following 5 stimuli: dural indentation, brushing, pressuring or pinching the skin, and brushing the cornea. Mean firing rates of respective values were compared using nonparametric statistics (Wilcoxon signed-ranks test). Two-tailed level of significance was set at 0.05.

B. Results

The database for testing CGRP-mAb vs isotype-conAb effects on spontaneous and induced activity of naive trigeminovascular neurons consisted of 63 neurons. Of these, 31 were classified as WDR and 32 as HT. Of the 31 WDR neurons, 18 (11 in males, 7 in females) were tested before and after administration of the CGRP-mAb, and 13 (7 in males, 6 in females) were tested before and after administration of the isotype-conAb. Of the 32 HT neurons, 18 (11 in males, 7 in female) were tested before and after administration of the CGRP-mAb, and 14 (8 in males, 6 in females) were tested before and after administration of the isotype-conAb.

The database for testing CGRP-mAb vs. isotype-conAb effects on the activation and sensitization of the neurons by CSD consisted of 50 neurons. Of these, 23 were classified as WDR and 27 as HT. Of the 23 WDR neurons, 13 (7 in males, 6 in females) were tested in the CGRP-mAb treated animals and 10 (5 in males, 5 in females) in the isotype-conAb treated animals. Of the 27 HT neurons, 14 (8 in males, 6 in female) were tested in the CGRP-mAb treated animals, and 13 (7 in males, 6 in females) in the isotype-conAb treated animals. Recording sites, receptive fields and neuronal classes.

Recording site, maps of dural and cutaneous receptive fields, and cell types did not differ between neurons tested for CGRP-mAb and those tested for the isotype-conAb. All identified recording sites were localized in laminae I-II and IV-V of the first cervical segment of the spinal cord and the caudal part of nucleus caudalis. In all cases, the most sensitive area of the dural receptive field was along the transverse sinus and the most sensitive area of the cutaneous receptive field was around the eye, involving the cornea in more than 90% of the cases. Spontaneous activity of naive central trigeminovascular neurons.

In male rats, intravenous administration of the CGRP-mAb reduced the spontaneous activity of the HT but not the WDR neurons. In the HT group, neuronal firing decreased within 3-4 hrs by 90% (p=0.040). Occasionally, the firing rate of some HT neurons decreased within 1-2 hours after the intravenous administration of the CGRP-mAb. In contrast, intravenous administration of the isotype-conAb did not alter the spontaneous activity of either group of neurons.

In females, unlike in males, intravenous administration of the CGRP-mAb did not reduce the spontaneous activity of HT or WDR neurons. Similarly, intravenous administration of the isotype-conAb did not alter the spontaneous activity of either group of neurons. Critically, the baseline (i.e., before any treatment) spontaneous firing rate of HT and WDR neurons did not differ between the male and the female rats (p=0.14).

For the HT neurons, mean spikes/sec before any treatment was 1.7±1.1 in the male vs. 1.9±1.0 in the female (p=0.55). For the WDR neurons, mean spikes/sec before any treatment was 0.3±0.6 in the male vs. 2.2±1.1 in the female (p=0.16).

Sensitivity of Naive Central Trigeminovascular Neurons to Dural Indentation

In both male and female rats, intravenous administration of the CGRP-mAb reduced the sensitivity to mechanical stimulation of the dura in the HT but not the WDR neurons. In males, the firing of HT neurons decreased by 75% (p=0.047) whereas in females it decreased by 61% (p=0.017). Regardless of the sex, intravenous administration of the isotype-conAb did not alter the sensitivity to dural stimulation in either group of neurons. Sensitivity of naive central trigeminovascular neurons to mechanical stimulation of the periorbital skin and the cornea. Intravenous administration of the CGRP-mAb- or the isotype-conAb did not alter the responses of HT or WDR neurons to innocuous (brush, pressure) or noxious (pinch) mechanical stimulation of the skin or the cornea in male or female rats.

Cortical Spreading Depression

Effects of CGRP-mAb (n=27) or isotype-conAb (n=23) on activation of central trigeminovascular neurons by CSD was tested in 50 neurons in which baseline firing rate (i.e., mean spikes/sec before induction of CSD) was reliable and consistent over hours. At baseline (i.e., before CSD), the spontaneous firing rate of HT and WDR neurons did not differ between the male and the female rats (p=0.14). For the HT neurons, mean spikes/sec before induction of CSD was 1.2±0.6 in the male vs. 3.3±1.7 in the female (p=0.29). For the WDR neurons, mean spikes/sec before induction of CSD was 1.5±0.6 in the male vs. 3.5±2.2 in the female (p=0.37).

CSD-Induced Activity in Central Trigeminovascular Neurons

In male rats, two hours after induction of CSD and 6 hours after isotype-conAb administration, the mean firing rate of the 7 HT neurons increased from 1.1±0.8 spikes/sec before CSD to 10.2±2.1 after CSD (p=0.019), whereas the mean firing rate of the 5 WDR neurons did not increase (0.5±0.3 spikes/sec before CSD vs. 1.6±0.5 after CSD; p=0.14). In contrast, in the CGRP-mAb treated rats, the response magnitude of the 8 HT neurons remained unchanged 2 hours after induction of CSD and 6 hours after CGRP-mAb administration (1.2±0.6 spikes/sec before CSD vs. 1.9±1.5 after CSD, p=0.29). In other words, the expected CSD-induced activation of the HT neurons was prevented by the CGRP-mAb treatment.

In female rats, two hours after induction of CSD and 6 hours after isotype-conAb administration, the mean firing rate of the 6 HT neurons increased from 1.9±1.0 spikes/sec before CSD to 10.0±4.5 after CSD (p=0.027), whereas the mean firing rate of the 5 WDR neurons remained unchanged (2.6±1.2 spikes/sec before CSD vs. 2.2±0.9 after CSD p=0.73). In contrast, in the CGRP-mAb treated rats, the response magnitude of the 6 HT neurons remained unchanged 2 hours after induction of CSD and 6 hours after CGRP-mAb administration (3.3±1.7 spikes/sec before CSD vs. 5.0±3.4 after CSD, p=0.45). As in the male, the expected CSD-induced activation of the HT neurons was prevented by the CGRP-mAb treatment. To further examine CGRP-mAb effects on the activation of WDR and HT neurons by CSD, a case-by-case analysis was also performed. Of all CGRP-mAb and isotype-conAb treated WDR neurons, 5/13 and 4/10 were activated by CSD, a mere 2% difference. In contrast, of all CGRP-mAb and isotype-conAb treated HT neurons, 2/14 and 13/13 were activated by CSD, an 86% difference.

CSD-Induced Sensitization

Regardless of activation by CSD, 11/13 HT and none of the WDR neurons fulfilled criteria for the development of sensitization (defined in the data analysis section). Therefore, the CGRP-mAb's ability to interfere with the development of sensitization after CSD is presented for HT but not WDR neurons. Expansion of dural receptive fields and enhanced responses to mechanical stimulation of the dura after CSD. In the isotype-conAb treated group, dural receptive fields expanded in 5/7 HT neurons in males and 6/6 HT neurons in females. Two hours after induction of CSD (6 hours after isotype-conAb administration), neuronal responses to dural indentation with VFH increased in all 7 HT neurons in the male (12.8±3.9 spikes/sec before CSD vs. 22.0±3.7 after CSD; p=0.026), and all 6 HT neurons in the female (8.5±1.7 before CSD vs. 21.6±5.1 after CSD, p=0.047).

In contrast, in the CGRP-mAb treated group, expansion of dural receptive fields, which was smaller when it occurred, was recorded in only 2/8 HT neurons in the male and 0/6 in the female. Two hours after induction of CSD (6 hours after CGRP-mAb administration), neuronal responses to dural indentation with VFH remained unchanged in all HT neurons in both the male (1.8±0.6 before CSD vs. 1.9±1.5 after CSD, p=0.83) and the female (10.5±1.6 before CSD vs. 8.1±6.4 after CSD, p=0.72)—indicative of prevention of sensitization. Thus, the CGRP-mAb prevented the development of intracranial mechanical hypersensitivity in HT neurons in both male and female rats. Expansion of cutaneous receptive fields and enhanced responses to mechanical stimulation of the periorbital skin after CSD (i.e., central sensitization).

In the isotype-conAb treated group, facial receptive fields expanded in 5/7 HT neurons in males and 6/6 HT neurons in females. Two hours after induction of CSD (6 hours after isotype-conAb administration) responses to brush and pressure increased significantly in all 13 HT neurons (7 in males, 6 in females). In males, responses to brush and pressure increased from 0.0 to 18.2±9.1 spikes/sec (p=0.046) and from 16.6±4.2 to 35.8±9.1 spikes/sec (p=0.045), respectively. In females, responses to brush and pressure increased from 0.0 to 8±6.5 spikes/sec (p=0.027) and from 9.3±2.7 to 31.8±13.6 spikes/sec (p=0.016), respectively. In contrast, responses to pinch increased significantly in all HT neurons in females (19.3±5.0 spikes/sec before CSD vs. 45.8±12.4 spikes/sec after CSD, n=6, p=0.027) but not in the male (33.8±7.1 spikes/sec before CSD vs. 52.4±10.3 spikes/sec after CSD, n=6, p=0.068).

In the CGRP-mAb treated rats, facial receptive fields expanded in only 2/8 HT neurons in males and 0/6 HT neurons in females. Two hours after induction of CSD (6 hours after CGRP-mAb administration), neuronal responses to brush (p=0.35), pressure (p=0.63) and pinch (p=0.78) remained unchanged in all HT neurons in both male and female—suggesting that the CGRP-mAb prevented induction of sensitization.

Enhanced Responses to Corneal Stimulation after CSD

In the isotype-conAb treated rats, responses to corneal stimulation after CSD increased significantly in females (7.6±1.9 spikes/sec before CSD vs. 21.0±6.4 spikes/sec after CSD, n=6, p=0.044) but not in males (1 1.0±2.6 spikes/sec before CSD vs. 21.6±8.7 spikes/sec after CSD, n=7, p=0.19) HT neurons. In the CGRP-mAb treated female rats, response to brushing the cornea remained unchanged in the 6 HT neurons (p=0.51)—suggesting prevention of sensitization; and as expected, it also remained unchanged in the 8 HT neurons in the males (10.8±3.3 spikes/sec before CSDS vs. 9. ±1.8 (spikes/sec after CSD, p=0.60). Thus, the CGRP-mAb prevented the development of corneal hypersensitivity in HT neurons in female but not male rats.

C. Discussion

The study demonstrates that the humanized monoclonal anti-CGRP antibody fremanezumab inhibits activation and sensitization of HT but not WDR trigeminovascular neurons. In males, the CGRP-mAb inhibited the spontaneous activity of naive HT neurons and their responses to stimulation of the intracranial dura but not facial skin or cornea, whereas in females it only inhibited their responses to stimulation of the intracranial dura. When given sufficient time, however, the CGRP-mAb prevented in both sexes the activation and consequential sensitization of the HT neurons by CSD, but not the partial activation of WDR neurons. Mechanistically, these findings suggest that HT neurons play a critical role (not recognized before) in the initiation of the perception of headache and the development of allodynia and central sensitization. Clinically, the present findings may help explain the therapeutic effectiveness of CGRP-mAb in preventing headaches of intracranial origin such as migraine and why this therapeutic approach may not be effective for every migraine patient.

This study tested the effects on CGRP-mAb on the responsiveness of different classes of central trigeminovascular neurons. Previously, Storer and colleagues showed that the CGRP-R antagonist BIBN4096BS inhibits naive central trigeminovascular neurons responses to electrical stimulation of the superior sagittal sinus and microiontophoretic administration of L-glutamate (Storer et al, 2004, Br. J. Pharmacol. 142: 1171-1181).

Fremanezumab Effects on HT Vs. WDR

When given intravenously, CGRP-mAb reduced baseline spontaneous activity in HT but not WDR neurons. Considering current and previous evidence that WDR trigeminovascular neurons are activated by a variety of dural stimulation used to study the pathophysiology of migraine (Davis and Dostrovsky, 1988, J. Neurophysiol. 59:648-666; Burstein et al, 1998, J. Neurophysiol. 79:964-982; Storer et al, 2004, Brit. J. Pharmacol. 142: 1171-1181; Zhang et al, 2011, Ann. Neurol. 69: 855-865), it is reasonable to conclude that activation of WDR alone is insufficient to induce the headache perception in episodic migraine patients whose headaches are completely or nearly completely prevented by CGRP-mAb therapy (Bigal et al, 2015, Lancet Neurol. 14: 1081-1090). Conversely, it is also reasonable to speculate that activation of WDR trigeminovascular neurons alone may be sufficient to induce the headache perception in those episodic migraine patients who do not benefit from CGRP-mAb therapy, as the headache could be unaffected by elimination of the signals sent to the thalamus from HT trigeminovascular neurons. Outside migraine and the trigeminovascular system, HT and WDR neurons have been thought to play different roles in the processing of noxious stimuli and the perception of pain (Craig A D, 2002, Nat. Rev. Neurosci. 3:655-666; Craig A D, 2003, Trends Neurosci. 26:303-307; Craig A D, 2003, Annu. Rev. Neurosci. 26: 1-30). While most HT neurons exhibit small receptive fields and respond exclusively to noxious mechanical stimuli, most WDR neurons exhibit large receptive fields and respond to both mechanical and thermal noxious stimuli (Price et al, 1976, J. Neurophysiol. 39:936-953; Price et al, 1978, J. Neurophysiol. 41:933-947; Hoffman et al, 1981, Neurophysiology 46:409-427; Dubner and Bennett, 1983, Annu. Rev. Neurosci. 6: 381-418; Bushnell et al, 1984, J. Neurophysiol. 52: 170-187; Surmeier et al, 1986, J. Neurophysiol. 56:328-350; Ferrington et al, 1987, J. Physiol. (Lond) 388:681-703; Dubner et al, 1989, J. Neurophysiol. 62:450-457; Maixner et al, 1989, J. Neurophysiol. 62:437-449; Laird and Cervero, 1991, J. Physiol. 434:561-575). Based on these differences, it is generally believed that HT neurons make a greater contribution to the spatial encoding (size, location) of pain and a lesser contribution to the encoding of pain modalities, whereas WDR neurons make a greater contribution to the radiating qualities of the pain. Along this line, it is also reasonable that those patients unresponsive to fremanezumab are the ones whose headaches affect large areas of the head (i.e., frontal, temporal, occipital, bilateral) whereas the ones whose headaches are well localized to small and distinct areas will be among the responders.

Effectiveness in Headache

Fremanezumab reduced responsiveness to mechanical stimulation of the dura (both in males and females) but not to innocuous or noxious stimulation of the skin or cornea. This finding, together with the fact that the CGRP-mAb also prevented the activation of HT trigeminovascular neurons by CSD, provides a scientific basis for fremanezumab's effectiveness in preventing headaches of intracranial origin. Conversely, lack of effects on modulating the processing of sensory and nociceptive signals that arise in the facial skin and cornea predicts that this class of drugs will have little therapeutic effect on treating prolonged trigeminal pain conditions such as dry eye and herpes-induced trigeminal neuralgia. Given that fremanezumab inhibited activation of central trigeminovascular neurons from the dura (mechanical, CSD) but not skin or cornea, and that the size of this molecule is too large to readily penetrate the blood brain barrier, it is reasonable to suggest that the inhibitory effects described above were secondary to (primary) inhibition of responses to dural indentation and CSD in peripheral trigeminovascualr neurons. Given the wide distribution throughout the body of CGRP fibers (Kruger et al, 1988, J. Comp. Neurol. 273: 149-162; Kruger et al, 1989, J. Comp. Neurol. 280:291-302; Silverman and Kruger, 1989, J. Comp. Neurol. 280:303-330), their presence in multiple spinal cord segments (Hansen et al., 2016, Pain 157:666-676; Nees et al., 2016, Pain 157:687-697), and in multiple sensory dorsal root ganglia (Edvinsson et al., 1998, J. Auton. Nerv. Syst. 70: 15-22; Edvinsson et al, 2001, Microsc. Res. Techniq. 53:221-228; Cho et al, 2015, J. Korean Med. Sci. 30: 1902-1910; Kestell et al, 2015, J. Comp. Neurol. 523:2555-2569; Spencer et al, 2016, J. Comp. Neurol. 524:3064-3083), it is surprising that the CGRP-mAb had little or no effect on the responses of the central neurons to noxious stimulation of the skin and cornea. If one accepts the notion that the CGRP-mAb acts mainly in the periphery, it is also reasonable to propose that peripheral aspects of the sensory innervation of the meninges and the way this innervation affects sensory transmission in the dorsal horn differ from those involved in the generation of cutaneous, corneal or other (somatic) pains. Studies on fremanezumab's effects in animal models of other pain conditions should allow for more accurate interpretation of the difference between the CGRP-mAb's effects in the dura vs. extracranial tissues not believed to have a distinct initiating role in migraine.

Inhibition of CSD-Induced Activation and Sensitization

This study demonstrates sensitization of central trigeminovascular neurons by CSD. This sensitization—observed in HT but not WDR neurons in both males and females—was prevented by the CGRP-mAb administration. These findings indicate that cutaneous allodynia in attacks preceded by aura (Burstein et al, 2000, Ann. Neurol. 47:614-624) is mediated by HT neurons that are unresponsive to innocuous mechanical stimulation of the skin at baseline (interictally in patients and before induction of CSD in animals), but become mechanically responsive to brush after the CSD. According to this scenario, among migraine aura patients, responders to the prophylactic treatment with CGRP-mAb would show no signs of cutaneous allodynia.

Male v. Female

This study also tested CGRP-mAb's effects in both male and female rats. While the overall analysis-by-sex suggests that the therapeutic benefit of this class of drugs should be similar in male and female migraineurs, it also shows that in the naive state, CGRP-mAb reduces the spontaneous activity in male, but not female HT neurons, and that after induction of sensitization by CSD, only HT neurons recorded in females exhibited signs of sensitization to noxious stimulation of the skin and cornea. Given that migraine is more common in women than men, the differences may suggest that hyperalgesia (rather than allodynia) is more likely to develop in women than in men during migraine with aura, and that attempts to reduce neuronal excitability by CGRP-mAb in the interictal state (i.e., as a preventative), may also be more challenging in women than men. Mechanistically, the three observed differences could be attributed to greater excitability of female HT neurons, either due to these neurons' internal properties or due to differences in the strength of inputs they receive from peripheral nociceptors. Whereas no data exist to support the first option, it is possible that differences in the activation of dural immune cells and inflammatory molecules in females compared to males (Mcllvried et al. (2015) Headache 55:943-957) can support the second option. Regarding fremanezumab's ability to reduce spontaneous activity in male but not female rats, one may take into consideration data showing that female rats express fewer CGRP receptors in the trigeminal ganglion and spinal trigeminal nucleus, and higher levels of CGRP-encoding mRNA in the dorsal horn (Stucky et al. (2011) Headache 51:674-692).

Finally, the inhibitory effects of CGRP-mAb required only a few hours to reach significance. This relatively short time (hours rather than days) was achieved using intravenous administration.

### Example 2. Assessing Anti-CGRP Antibody (TEV-48125) Responders Using Behavioral and Psychophysical Tools

The majority of episodic migraineurs seeking secondary or tertiary medical care exhibit signs of cutaneous allodynia and hyperalgesia during the acute phase of migraine, but not when pain-free (Burstein R et al. (2000) Ann. Neurol. 47:614-624). In contrast, chronic migraine patients commonly exhibit sign of cutaneous allodynia and hyperalgesia both during acute migraine attacks as well as during the interictal phase. Mechanistically, allodynia and hyperalgesia are thought to be mediated by sensitization of central trigeminovascular neurons in the spinal trigeminal nucleus (Burstein R et al. (1998) J. Neurophysiol. 79(2): 964-982; Burstein R et al. (2000) Ann. Neurol. 47: 614-624; and Lipton et al. (2008) Ann. Neurol. 63(2): 148-58). In contrast, chronic migraine patients commonly exhibit sign of cutaneous allodynia and hyperalgesia both during acute migraine attacks as well as during the interictal phase. Mechanistically, allodynia and hyperalgesia are thought to be mediated by sensitization of central trigeminovascular neurons in the spinal trigeminal nucleus (see Burstein (1998)). Example 5 demonstrates that TEV-48125, through its inhibitory action in peripheral meningeal nociceptors, is capable of preventing the activation and sensitization of high-threshold (HT) neurons in the spinal trigeminal nucleus to an extent that is far superior than its ability to inhibit wide-dynamic range (WDR) neurons (see also Melo-Carrillo et al. (2017) J. Neurosci. 37(30): 7149-63). Given that HT neurons respond exclusively to noxious (painful) stimuli whereas WDR neurons respond preferentially to noxious stimuli (i.e., their response to noxious stimuli is larger than their response to innocuous stimuli), it is reasonable to hypothesize that the blockade of HT will prevent hyperalgesia more effectively than allodynia.

To date, there are no examples or hints in the literature of examples of drugs that reduce activation and sensitization of only one of these two classes of nociceptive neurons in the spinal trigeminal nucleus. Given that fremanezumab inhibits meningeal Aδ- but not C-fibers, the selective inhibition of the Aδ-fibers potentially explains the antibody's selective inhibition of HT neurons (see Melo-Carillo et al. (2017) J. Neurosci. 37(44): 10587-96). Also, since C-fibers may not influence the activity of HT neurons, consequently, fremanezumab may achieve a very selective effect on ascending nociceptive trigeminovascular pathways—those whose activity depends on CGRP release in the periphery.

Without wishing to be bound by any particular theory, it is believed that responders are subjects in which ongoing peripheral input is required to maintain the central sensitization in WDR and HT neurons, whereas non-responders are subjects in which ongoing peripheral input is not required to maintain the central sensitization in WDR and HT neurons. Since fremanezumab blocks activation of the Aδ-fibers, in responders this blockade may be sufficient to render HT neurons completely quiescent (i.e., terminate their sensitization). Fremanezumab may also decrease the overall input that drives the sensitization state of the WDR neurons to the extent that the input that the neurons receive from the unblocked C-fibers only induces excitatory post-synaptic potentials (EPSPs), but not actual action potentials. The sensitization state of both WDR and HT neurons may be reversed by fremanezumab and consequently, the allodynia/hyperalgesia will be reversed in the responders. Conversely, in non-responders, the sensitization of either HT or WDR neurons, or both, is completely independent of the peripheral input, regardless of whether it originates in the Aδ- or C-fibers. Accordingly, the non-responders will be allodynic and/or hyperalgesic after treatment. It is expected that other anti-CGRP active agents (e.g., a gepant as described herein) will exhibit the same behavior as fremanezumab.

Study Design:

Overall strategy: To determine cutaneous pain thresholds (which test for allodynia), and pain rating in response to repeated suprathreshold mechanical and heat stimuli (which test for hyperalgesia) in chronic migraine patients under 4 different conditions: (a) before treatment while migraine-free, (b) after treatment while migraine-free, and if possible, (c) before and after treatment while in the middle of acute migraine attack. Note: part (c) is not necessary for identifying responders among the CM population. It may be relevant to identifying responders among the high-frequency episodic patients.

Participant selection and recruitment: Individuals with chronic migraine will be considered for participation in this study. Primary inclusion criteria will be (1) age 18-64 years old, (2) history of chronic migraine with or without aura, based on the International Classification of Headache Disorders (3rd edition) for at least 3 years, and (3) ability to communicate in English (in order to understand and follow instructions of testing). Exclusion criteria will include: (1) less than fifteen headache days per month; (2) pregnancy; (3) history of coronary artery bypass surgery, heart attack, angina, stroke, serious gastrointestinal bleeding, peptic ulcer disease; or chronic kidney disease; (5) having medical conditions requiring use of diuretics or daily anticoagulants.

Open-label design: After screening, which will be performed on a pre-scheduled day (visit 1), the migraine history of study participants will be captured using a questionnaire, and quantitative sensory testing for allodynia and hyperalgesia will be performed. Visit 1 will take place at least 30 days prior to visit 2, when the participant is headache-free. Participants will be instructed to maintain a daily headache diary during this period.

Visit 2 will take place when the study participant has migraine, and will include 3 cycles of pain rating and QST for the evaluation of allodynia and hyperalgesia. The first cycle of pain rating will take place prior to treatment and at least 2 hours after attack onset. Patients will be randomized to receive either placebo or 75 mg of rimegepant orally. The second cycle of pain rating will take place two hours after treatment. The third cycle of pain rating will take place 4 hours after treatment.

Participants will be instructed to maintain a daily headache diary throughout the study.

Visit 3 will take place 1 week after treatment and will include headache diary review, rating of headache intensity, and QST testing for allodynia and hyperalgesia.

Visit 3 will take place 4 weeks after treatment and will include headache diary review, rating of headache intensity, and QST testing for allodynia and hyperalgesia. In each visit, the baseline headache intensity, pain threshold to quantitative mechanical and thermal stimuli, and headache intensity score in response to suprathreshold mechanical and heat stimuli will be documented.

Quantitative Sensory testing (QST): Testing will be done in a quiet room away from noise and distraction. Patients will be able to choose their most comfortable position (sitting on a chair or laying in bed) during the sensory testing. In each testing session, pain thresholds to hot and mechanical stimulation will be determined in the skin over the site to where the pain is referred to. This site includes most commonly the periorbital and temporal regions. Heat skin stimuli will be delivered through a 30×30 mm² thermode (Q-Sense 2016, Medoc) attached to the skin at a constant pressure and their pain thresholds will be determined by using the Method of Limit.

Allodynia testing: To determine pain thresholds, the skin will be allowed to adapt to a temperature of 32° C. for 5 minutes and then warmed up at a slow rate (1° C./sec) until pain sensation is perceived, at which moment the subject stops the stimulus by pressing a button on a patient response unit. Heat stimuli will be repeated three times each and the mean of recorded temperatures will be considered threshold. Pain threshold to mechanical stimuli will be determined by using a set of 20 calibrated von Frey hairs (VFH, Stoelting). Each VFH monofilament is assigned a scalar number in an ascending order (1=0.0045 g, 2=0.023 g, 3=0.027 g, 4=0.07 g, 5=0.16 g, 6=0.4 g, 7=0.7 g, 8=1.2 g, 9=1.5 g, 10=2.0 g, 11=3.6 g, 12=5.4 g, 13=8.5 g, 14=11.7 g, 15=15.1 g, 16=28.8 g, 17=75 g, 18=125 g, 19=28 Ig). Because a linear relationship exists between the log force and the ranked number, mechanical pain thresholds are expressed as VFH numbers (#) rather than their forces (g). Each monofilament will be applied to the skin 3 times (for 2 sec) and the smallest VFH number capable of inducing pain at two out of three trials will be considered threshold. Skin sensitivity will also be determined by recording the subject's perception of soft skin brushing, which is a dynamic mechanical stimulus, as distinguished from the VFH, which is a static mechanical stimulus, as distinguished from the VFH, which is a static mechanical stimulus.

Hyperalgesia testing: When a painful stimulus is perceived as more painful than usual, the subject is considered hyperalgesic. To determine whether the subject is hyperalgesic, 3 supra-threshold heat and mechanical stimuli will be applied to the skin. The value of the supra-threshold stimulus will be determined during the allodynia testing above. For example, if the heat pain threshold is 45° C., we will 46° C. in the hyperalgesia testing. In this test, the skin will be exposed to 3 supra-threshold stimuli (1-above-threshold), each lasting 10 seconds and separated by 10 seconds (i.e., inter-stimulus interval of 10 seconds). At the end of each stimulus, the patient will have 10 seconds to identify the intensity of the pain using a visual analog scale (VAS) of 0-10 (o=no pain, 10=most imaginable pain). Similar test will be administered using supra-threshold mechanical stimulation.

The equipment used for quantitative sensory testing has FDA approval. It is routinely used by neurologists, nurses, and pain specialists throughout the country. It imposes no risk or discomfort, and since it is controlled by the patient, stimuli can be stopped at any time.

Interpretation of QST:

Allodynia: Since the detection of pain thresholds depends on subjective data input, several algorithms have been developed in order to minimize subjective variation, and make the results as objective as possible. These algorithms are incorporated into the software program that controls the thermal and mechanical sensory analyzer (0-Sense 2016). In healthy subjects, pain thresholds for heat and mechanical skin stimuli range between 42-47° C. and 75-281 g, respectively (see Lindblom (1994) Analysis of abnormal touch, pain, and temperature sensation in patients. In: Boivie J, Hansson P, Lindblom U, eds. Touch, temperature and pain in health and disease: mechanism and assessments. Vol, 3. Progress in brain research and management. Seattle: IASP press, p 63-84; and Strigo et al. (2000) Anesthesiology 92(3): 699-707. Using a more stringent criteria, a subject will be considered to be allodynic if her/his pain threshold is below 41° C. for heat, and below 30 g for skin indentation with the calibrated von Frey hairs. Meeting the criterion for any one modality will be sufficient to determine that the subject is allodynic (Burstein et al. (2004) Ann. Neurol. 47(5): 614-24; and Burstein et al. (2004) Ann. Neurol. 55(1): 19-26.(

Hyperalgesia: Any change in pain rating that is larger than 30% will be considered as evidence for hyperalgesia (e.g., if supra-threshold stimulus #1 is rated 6/10 on a VAS, supra-threshold stimulus #3 will have to be rated at 8/10 or higher).

Data analysis will take into consideration values of mechanical and heat pain thresholds before and after treatment.

Data Analysis:

Data analyses will include subjects who complete all 4 visits and 6 testing sessions.

The primary outcome measure is the presence or absence of allodynia after the intervention (1 month) in responders vs. non-responders. Responders are primarily defined as experiencing a minimal reduction of 50% in monthly headache days; non-responders are defined as experiencing a maximal reduction of less than 50% in monthly headache days. A secondary definition addresses responders as experiencing a minimal reduction of 60% in monthly headache days; non-responders are defined as experiencing a maximal reduction of less than 40% in monthly headache days. An additional secondary definition addresses responders as experiencing a minimal reduction of 75% in monthly headache days; non-responders are defined as experiencing a maximal reduction of less than 25% in monthly headache days. The proportion of responders found to have an absence of allodynia and/or hyperalgesia when the participant is headache-free (the interictal phase of the migraine) is significantly higher in the population of responders who are found to have allodynia and/or hyperalgesia when headache-free.

The primary outcome measure will be examined using a Chi-square (χ²) test to assess the categorical association between the presence of allodynia (yes/no) and the responsiveness of subjects (yes/no). Secondary outcome measures are migraine duration (hours) before and after the intervention (1 month) and changes in headache intensity at 2 and 4 hours after intervention.

Data of the continuous secondary outcome measures will first be tested for normality so as to determine whether parametric or non-parametric analyses are appropriate.

Accordingly, parameters of central distribution (means/medians) will be used to assess differences in these variables between responders and non-responders.

Analyses will also examine the effects the following factors on the primary and secondary outcome measures: number of years with migraine, number of years with CM, family history, associated symptoms (e.g., nausea, vomiting, photophobia, phonophobia, osmophobia, aura, muscle tenderness), common triggers (e.g., stress, prolong wakefulness food deprivation, menstruation), and acute as well as prophylactic treatment history.

Power Analysis:

Power analysis was based on the Chi-square (χ²) Goodness-of-Fit and Z comparison of proportions tests. Incorporated were a of 5% (significance level), I-β error probability of 90% (power), w of 0.36 (effect size; χ² Goodness-of-Fit test), and allocation ratio of 1:1 (Z comparison of proportions test). Stratification analysis included the variables Group (placebo vs. treatment), Responsiveness (Responder vs. Non-responder; see definition above), and Allodynia (Presence vs. Absence). The primary hypothesis was that post-intervention proportions of Responders (according to the aforementioned definition of the 50% reduction threshold in monthly headache days) in the treatment and placebo groups would be 55% and 25%, respectively (based on published data by Bigal et al. (2015) Lancet Neurol. 14(11): 1091-100). This computation yielded a required number of 64 subjects in each of the placebo and treatment groups (df=5; critical χ<2>=11.07; noncentrality parameter λ=16.51. An additional 20% were accounted for potential dropout. Thus, a total of 77 patients are to be enrolled in each group, yielding a total of 144 patients in the entire study.

### Example 3: A Clinical Study of Anti-CGRP Agent Responder Rate

Twenty-nine high frequency episodic migraine, anti-CGRP agent naïve, patients underwent QST testing at least twenty-four hours into the post-ictal phase of their migraine and completed a thirty-day e-diary questionnaire before initiation of therapy with an anti-CGRP agent. After three months of treatment with the anti-CGRP agent, the patients were reviewed to determine whether they had responded to the agent. Effective treatment was determined by a number of factors including reductions in headache intensity and the frequency of headaches, throbbing, photophobia, phonophobia and nausea. Responders were defined as those patients reaching at least a 50% reduction in monthly average number of migraine days during the 3-month treatment period. Following determination of response to the treatment, the findings were compared together with the previously blinded allodynia/hyperalgesia assessments. For those patients who demonstrated allodynia and/or hyperalgesia in QST testing at least twenty-four hours into the post-ictal phase of the migraine, completion of the allodynia and/or hyperalgesia determination was made based on e-diary questionnaire answers when the patients were migraine free for at least seventy-two hours.

The results of the study are shown in Table 1 and demonstrate that those patients who did not present with allodynia and/or hyperalgesia were significantly more likely to respond to the anti-CGRP agent therapy than those who did present with allodynia and/or hyperalgesia.

TABLE 1

Clinical Outcome

| | Non-Responder | Responder |
|---|---|---|
| Non-allodynic/hyperalgesic | 1 | 17 |
| allodynic/hyperalgesic | 13 | 2 |

All patents, patent applications, and publications mentioned in this document are herein incorporated by reference to the same extent as if each individual publication was specifically and individually indicated to be incorporated by reference.

1-13. (canceled)

14. A method of treating migraine in a subject comprising:
a) determining or having determined whether the subject exhibits allodynia and/or hyperalgesia during the ictal phase of a migraine, and
b) administering a gepant to the subject that does not exhibit signs of allodynia and/or hyperalgesia during the interictal phase of the migraine.

15. The method of claim 14, wherein the subject suffers from episodic migraine.

16. The method of claim 14, wherein the subject suffers from chronic migraine.

17. The method of claim 14, wherein the subject is known to exhibit allodynia and/or hyperalgesia during an acute phase of the migraine.

18. The method of claim 14, wherein the subject was determined during the interictal phase of the migraine to have a heat pain threshold of above 41° C.

19. The method of 14, wherein the absence of allodynia and/or hyperalgesia during the interictal phase of the migraine was determined by quantitative sensory testing (QST).

20. The method of claim 14, wherein the absence of allodynia and/or hyperalgesia during the interictal phase of the migraine was determined by questionnaire.

21. The method of claim 14, wherein the gepant is selected from the group consisting of rimegepant, ubrogepant, vazegepant, atogepant, olcegepant, telcagepant, BI 44370 and MK-3207.

22. The method of claim 14, wherein the gepant is administered while the patient is migraine-free.

23. The method of claim 14, wherein the allodynia is cutaneous allodynia.

24. A method for reducing migraine frequency in a subject suffering from migraine comprising determining or having determined whether said subject exhibits, or does not exhibit, allodynia and/or hyperalgesia during an interictal phase of a migraine, and administering to said subject that does not exhibit signs of allodynia and/or hyperalgesia during the interictal phase of the migraine a gepant.

25. (canceled)

26. A method of treating migraine in a subject comprising: a) determining or having determined whether the subject exhibits allodynia and/or hyperalgesia during the interictal phase of a migraine, and b) administering a gepant to the subject that does not exhibit signs of allodynia and/or hyperalgesia during the ictal phase of the migraine.

27. (canceled)

28. The method of claim 26, wherein the gepant is administered within 3 hours of the start of the ictal phase of the migraine.

29-30. (canceled)

31. The method of claim 14, wherein the subject was determined during the interictal phase of the migraine to have a mechanical pain threshold of above 30 g for skin indentation with calibrated von Frey hairs.

32. The method of claim 14, wherein the gepant is administered within 3 hours of the start of the ictal phase of the migraine.

33. The method of claim 32, wherein the gepant is administered within 60 minutes of the start of the ictal phase of the migraine.

34. The method of claim 32, wherein the treatment is acute treatment.

35. The method of claim 24, wherein the subject is known to exhibit allodynia and/or hyperalgesia during an acute phase of the migraine.

36. The method of claim 24, wherein the gepant is selected from the group consisting of rimegepant, ubrogepant, vazegepant, atogepant, olcegepant, telcagepant, Bl 44370 and MK-3207.

37. The method of claim 26, wherein the gepant is selected from the group consisting of rimegepant, ubrogepant, vazegepant, atogepant, olcegepant, telcagepant, Bl 44370 and MK-3207.
